(19) United States
(12) Patent Application Publication
(10) Pub. No.: US 2005/0074434 A1
(43) Pub. Date: Apr. 7, 2005

(54) Remedies for metabolic bone diseases

(76) Inventors: Tomoaki Hoshino, Fukuoka (JP); Koichi Yokota, Sakai-shi (JP); Yusuke Kawase, Hyogo (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 10/468,011

(22) PCT Filed: Feb. 25, 2002
(86) PCT No.: PCT/JP02/01662

(30) Foreign Application Priority Data
Feb. 23, 2001 (JP) 2001-49458

(51) Int. Cl. A61K 39/395

(52) U.S. Cl. 424/93.7; 424/130.1

(57) ABSTRACT

Novel remedies for metabolic bone diseases containing an IL-18 inhibitor as the active ingredient. These remedies exert a therapeutic effect by inhibiting the effect of overexpressed IL-18 which closely relates to the onset of osteoporosis. They are also useful as remedies for other metabolic bone diseases.

### TECHNICAL FIELD

“Metabolic bone disease” refers to diseases stemming from bone metabolism disorders such as osteoporosis, renal bone diseases, and rheumatoid arthritis. The present invention relates to a therapeutic agent for metabolic bone disease comprising an interleukin-18 inhibitor as an active ingredient.

### BACKGROUND ART

Among metabolic bone diseases, in particular, a large number of patients suffer from osteoporosis, and that has become a social problem.

Osteoporosis can be defined as the condition of a disease in which the bone mass (mass of minerals mainly including calcium contained in bone) is reduced, and the microstructure of bone tissue is changed, so that the bones become fragile and susceptible to bone fracture. Osteoporosis can be classified into primary and secondary osteoporosis. The former refers to senile osteoporosis, for which no other cause than senility cannot be identified. The latter refers to osteoporosis whose cause is identified and that can be improved by removing that cause. However, in either osteoporosis, the molecular mechanism of its trigger and the genetic diathesis of the disease are not still clarified.

For treating osteoporosis, estrogens, active vitamin D3 derivatives, calcitonine, bisphosphonates, vitamin K, calcium or the like can be used, but the effect thereof is not satisfactory, and there is a demand for new therapeutic agents.

Interleukin-18 (IL-18) is a new cytokine that was discovered in 1995 as an interferon y (IFN-γ) inducer produced by macrophage [Nature 378,88-91 (1995)]. IL-18 is synthesized as a precursor (pro IL-18) and then cleaved by an interleukin-1β converting enzyme [caspase-1] or the like so as to be active (mature IL-18).

The precursor of a mouse IL-18 consists of 192 amino acids, and its mature IL-18 consists of 157 amino acids. The precursor of a human IL-18 consists of 193 amino acids, and its mature IL-18 consists of 157 amino acids. In this specification, “IL-18” refers to “mature IL-18”, unless otherwise specified.

The receptor of the IL-18 belongs to the IL-1 receptor family, and IL-18Rα and IL-18Rβ are known.

The IL-18 is known to act on type 1 helper T cells (Th1) and natural killer cells (NK cells) so as to induce the production of IFN-γ, and enhance cytotoxic activities by enhancing cytotoxic T cell activities, and is regarded as inflammatory cytokine that causes a Th1 response.

In connection with bone metabolism, it is reported that osteoblasts produce IL-18, and suppresses formation of osteoclasts (Udagawa et al., J Exp Med 1997 185:1005-1012), which suggests the possibility of treating osteoporosis using IL-18 itself.

### DISCLOSURE OF INVENTION

It is an object of the present invention to provide a novel therapeutic agent used for treating metabolic bone diseases, especially osteoporosis.

In order to clarify the role of IL-18 in vivo, the inventors of the present invention produced transgenic mouse (IL-18TG mouse) that produces IL-18 excessively and examined the effect thereof on bone.

As a result, compared with a non-transgenic mouse, the condition of osteoporosis was observed more in the IL-18TG mouse, and it was found that IL-18 was significantly involved in the formation of the condition of osteoporosis.

The present invention is related to therapeutic agents for metabolic bone disease, especially osteoporosis, comprising an IL-18 inhibitor as an active ingredient.

Hereinafter, the present invention will be described in detail.

### BRIEF DESCRIPTION OF DRAWINGS

FIG. 1 is a photograph of the cortical bone of the central portion of the femoral shaft of a hemizygous IL-18TG mouse. The length of the black line in FIG. 1 indicates 100 μm.

FIG. 2 is a photograph of the cortical bone of the central portion of the femoral shaft of a wild-type mouse littermate of the IL-18TG mouse of FIG. 1. The length of the black line in FIG. 2 indicates 100 μm.

### BEST MODE FOR CARRYING OUT THE INVENTION

There is no particular limitation regarding the IL-18 inhibitor used in the present invention, as long as it is a substance that suppresses the action of excessively expressed IL-18.

As the IL-18 inhibitor used in the present invention, for example, substances that inhibit conversion from a IL-18 precursor to a mature IL-18 can be used. A specific example of this substance is an inhibitor of cysteine protease. As the inhibitor of cysteine protease, interleukin-1β converting enzyme inhibitors (inhibitors of caspase-1) can be preferably used.

Furthermore, as the IL-18 inhibitor used in the present invention, substances that neutralize the activities of the IL-18 such as IL-18 binding protein and an anti-IL-18 antibody, and substances that inhibit the binding of the IL-18 to the IL-18 receptor can be used. Furthermore, inhibitors of signal transduction after IL-18 receptor binding can be used.

As the IL-18 inhibitor used in the present invention, in particular, substances that inhibit the binding of the IL-18 to the IL-18 receptor can be preferably used.

As the interleukin-1β converting enzyme inhibitor used in the present invention, various compounds are known, and specific examples thereof include peptide derivatives disclosed in Japanese Laid-Open Patent Publication (Kokai) No. 5-255218, sulfonamide derivatives disclosed in Japanese Laid-Open Patent Publication (Kokai) No. 11-147873, peptide derivatives disclosed in Japanese Laid-Open Patent Publication (Kohyo) No. 10-504285, glycine derivatives disclosed in Japanese Laid-Open Patent Publication (Kokai) No. 11-147895 and tetrazole derivatives disclosed in International Application WO97/24339.

The IL-18 binding protein can be prepared according to the method described in a reference [Immunity, 10, 127-136 (1999)].

A monoclonal antibody specific to the IL-18 can be prepared according to the method described in a reference [J. Immunol. Methods, 217, 97-102 (1998)].

A specific example of the substances that inhibit the binding of the IL-18 to the IL-18 receptor (IL-18Rα) is a IL-18 receptor protein and a monoclonal antibody specific to the IL-18 receptor.

The monoclonal antibody specific to the IL-18 receptor either can be an antibody derived from mammals, a chimeric antibody, or a humanized antibody.

The monoclonal antibody specific to the IL-18 receptor protein and the IL-18 receptor used in the present invention, for example, can be prepared according to the method described in Japanese Laid-Open Patent Publication (Kokai) No. 11-100400.

The therapeutic agent of the present invention for metabolic bone disease can be administered to patients as appropriate in various pharmaceutical forms such as pharmaceuticals for oral administration, injections, or inhalations.

Furthermore, the therapeutic agent of the present invention for metabolic bone disease can be used in combination with other pharmaceuticals used for treating osteoporosis such as estrogens as appropriate. In addition, the interleukin-1 converting enzyme inhibitors, IL-18 binding proteins, anti-IL-18 antibodies, monoclonal antibodies specific to the IL-18 receptor can be used in combination of two or more as appropriate.

The various pharmaceuticals of the therapeutic agent of the present invention for metabolic bone disease can be produced by a regular method.

Examples of dosage forms for oral administration include tablets, capsules, granules, fine granules and powders, and these pharmaceuticals are produced by a regular method by mixing, as appropriate, the IL-18 inhibitor used in the present invention and ordinary medical additives such as lactose, corn starch, crystalline cellulose, magnesium stearate, carboxymethylcellulose calcium, hydroxy propyl cellulose or talc.

Injections can be prepared by a regular method, and isotonizing agents such as mannitol, sodium chloride, glucose, sorbitol, glycerol, xylitol, fructose, maltose and mannose, stabilizing agents such as sodium sulfite and albumin, preservatives such as benzyl alcohol and methyl p-hydroxy benzoate, or the like can be added in the pharmaceutical as appropriate.

The injections can be prepared in the form of a lyophilized pharmaceutical that is to be dissolved at the time of use. The lyophilized pharmaceutical can be produced by a regular method, and isotonizing agents, stabilizing agents or preservatives as described above can be added in the pharmaceutical as appropriate.

Inhalations can be produced by a regular method, and can be prepared by dissolving or suspending the IL-18 inhibitor used in the present invention in a physiological saline solution, and adding as appropriate isotonizing agents such as mannitol, sodium chloride, glucose, sorbitol, glycerol, xylitol, fructose, maltose and mannose, stabilizing agents such as sodium sulfite and albumin, preservatives such as benzyl alcohol and methyl p-hydroxy benzoate.

When the IL-18 inhibitor used in the present invention is a monoclonal antibody specific to the IL-18 receptor, the therapeutic agent for metabolic bone disease of the present invention generally can be used in the form of an injection or an oral agent. Such an injection or inhalations can be produced by a regular method.

The dosage amount of the therapeutic agent of the present invention for metabolic bone disease is typically 0.1 mg to 1000 mg per day, and this amount is administered once or in 2-3 divided doses, as appropriate, depending on the type of the inhibitor used in the present invention, the administration route, the condition, the age and the weight of the patient or the like.

The effect of the present invention is evident from the following test example. For the IL-18TG mouse, the IL-18 concentration in blood plasma is significantly higher than that of the wild-type mouse (see Test Example 1), and the area and thickness of the femoral cortical bone are smaller (see Test Example 2). However, there is no difference between the two types in the weight of the mice. Therefore, it is indicated that the IL-18 acts on reduction of the bone mass directly or indirectly, and the IL-18 inhibitor is useful as a therapeutic agent for metabolic bone disease, especially osteoporosis.

### TEST EXAMPLE 1

Confirmation of Formation of Disease Condition of Osteoporosis in IL-18 Transgenic Mouse

(1) Test Method

A transgenic mouse that produces IL-18 excessively (IL-18TG mouse) was produced, and its bone specimen was compared with that of a non-transgenic mouse.

A complementary DNA (cDNA) which has a gene of signal peptide from the V-J2-C region of the mouse immunoglobulin K chain fused with the mouse mature IL-18 gene was produced by amplification by polymerase chain reaction (PCR) using cDNA of a precursor of the IL-18 as the template. This DNA was incorporated into pCR2.1 vector and digested with EcoRI. This DNA fragment encoding the IL-18 and the signal peptide was inserted in the EcoRI site of a pEpIgH vector encoding a human Ep enhancer and a mouse IgVH promoter and thus a plasmid for gene introduction was completed.

The plasmid was digested with XbaI and SaII before being injected to a fertilized egg, and thus a 4.4 kb DNA fragment was obtained. This DNA fragment was injected to a fertilized egg of a C57BL/6 mouse. The tail of the mouse that was born was cut at the age of four weeks, so that genome DNA was extracted. This was amplified by PCR using the following three PCR primers, and thus an IL-18 transgenic mouse was confirmed.

5′-AACCGGGCCCCTCTGCTAACCATG-3′
5′-GGAACAATGGAGACAGACACACTCCTGCTATGG-3′
5′-CACCTAACTTTGATGTAAGTTAGTGAGAGTGAACAT-3′

Then, the IL-18 transgenic mouse and the wild-type C57BL/6 mouse were crossbred so that a hemizygous IL-18 TG mouse was produced. As a result, as shown in Table 1, the male hemizygous IL-18 TG mouse has a significantly higher IL-18 concentration in blood plasma than the wild-type C57BL/6 mouse littermate.

TABLE 1
Comparison of IL-18 concentration in blood plasma

| | IL-18 concentration in blood plasma |
|---|---|
| mouse | (ng/ml) |
| hemizygous IL-18 TG mouse | 6.4 ± 1.2 |
| wild-type C57BL/6 mouse | less than measurement range |
| | (<0.256) |

N = 4, average ± standard error

The male hemizygous IL-18 TG mouse and the wild-type C57BL/6 mouse littermate were sacrificed at the age of 10 weeks, and the femurs were extracted. The bones were subjected to formalin fixation and then decalcified, and thus thin specimens were produced. The specimens were subjected to hematoxylin eosin stain and the state of the bones was observed.

(2) Test Results

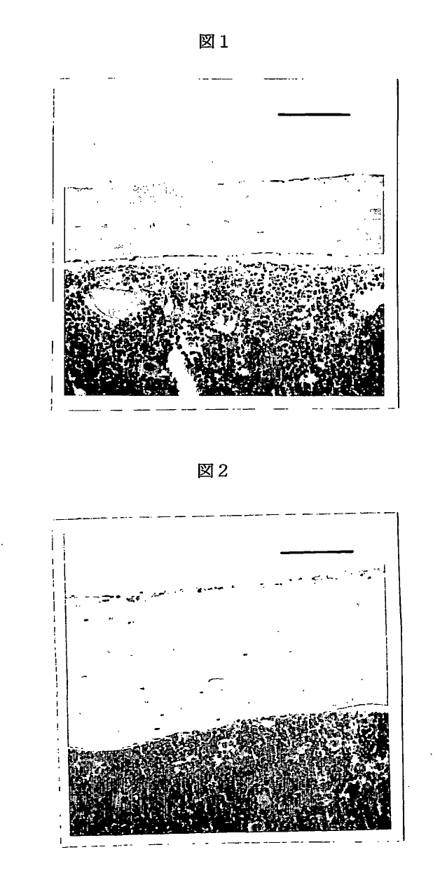

As shown in FIGS. 1 and 2, in the hemizygous IL-18 TG mouse bone specimen, a reduction in the mass of the bone was observed more, compared with the wild-type mouse.

### TEST EXAMPLE 2

Measurement of the State of the Femoral Cortical Bone of the IL-18 Transgenic Mouse

(1) Test Method

The left femur was removed from the male hemizygous IL-18 TG mouse and the male wild-type C57BL/6 mouse (at the age of 8 to 9 weeks) produced according to the method of Test Example 1. A thin specimen (a piece with a round cross-section obtained by vertically cutting the central portion of the femoral shaft; thickness of 3 μm) was produced by the same method, and the length of the outer circumference, the area and the thickness of the cortical bone were measured with image analysis software (Mac SCOPE, Mitani Corp.).

(2) Test Results

Table 2 shows the test results.

TABLE 2
Measurement of state of femoral cortical bone

mouse

| | hemizygous | wild-type |
|---|---|---|
| indicator | IL-18TG mouse | C57BL/6 mouse |
| outer circumferential | 4.51 ± 0.09 | 4.48 ± 0.14 |
| length (mm) | | |
| area (mm²) | 0.548 ± 0.025* | 0.631 ± 0.023 |
| thickness (mm) | 0.125 ± 0.004** | 0.158 ± 0.004 |

N = 6, mean ± standard error
Statistical analysis between IL-18 TG and wild-type mice was performed using the unpaired two-tailed Student t-test and the results were shown by a symbol * or **.
The symbol * indicates p < 0.05, and the symbol ** indicates p < 0.01.

The area and the thickness of the cortical bone of the central portion of the femoral shaft of the IL-18 TG mouse were smaller than those of the wild-type mouse.

Hereinafter, the present invention will be described more specifically by way of example.

### EXAMPLE 1

Injection:

Injections (5 mg/ampoule) containing a monoclonal antibody specific to a human IL-18 receptor were prepared by filtrating for sterilization PBS (1 mg/ml) in which a monoclonal antibody specific to a human IL-18 receptor was dissolved, and then pouring the same into ampoules in an amount of 5 ml per ampoule.

### Industrial Applicability

Among the therapeutic agent for the metabolic bone diseases of the present invention, the therapeutic agent for osteoporosis brings about a therapeutic effect by suppressing excessive expression of the activities of the IL-18 significantly involved in formation of the disease condition of osteoporosis.

1. A therapeutic agent for metabolic bone disease comprising an interleukin-18 inhibitor as an active ingredient.

2. The therapeutic agent according to claim 1, wherein the interleukin-18 inhibitor is a substance that inhibits conversion from an interleukin-18 precursor to a mature interleukin-18.

3. The therapeutic agent according to claim 1, wherein the interleukin-18 inhibitor is an inhibitor of cysteine protease.

4. The therapeutic agent according to claim 1, wherein the interleukin-18 inhibitor is a substance other than an interleukin-1β converting enzyme inhibitor.

5. The therapeutic agent according to claim 1, wherein the interleukin-18 inhibitor is an interleukin-18 binding protein.

6. The therapeutic agent according to claim 1, wherein the interleukin-18 inhibitor is a substance that neutralizes the activities of the interleukin-18.

7. The therapeutic agent according to claim 1, wherein the interleukin-18 inhibitor is a monoclonal antibody specific to an interleukin-18.

8. The therapeutic agent according to claim 1, wherein the interleukin-18 inhibitor is a substance that inhibits the binding of the interleukin-18 to the interleukin-18 receptor.

9. The therapeutic agent according to claim 1, wherein the interleukin-18 inhibitor is an interleukin-18 receptor protein or a monoclonal antibody specific to an interleukin-18 receptor.

10. The therapeutic agent according to claim 1, wherein the interleukin-18 inhibitor is an inhibitor of signal transduction after interleukin-18 binding to interleukin-18 receptor.

11. The therapeutic agent according to any one of claim 1 to 10, wherein the metabolic bone disease is osteoporosis.

12. An animal model of metabolic bone disease, which is introduced by interleukin-18 gene.

13. A method of screening a therapeutic agent for metabolic bone disease, which comprises using the animal model of metabolic bone disease of claim 12.
